(19) United States
(12) Patent Application Publication
(10) Pub. No.: US 2013/0035517 A1
(43) Pub. Date: Feb. 7, 2013

(54) PRODUCTION OF ALCOHOLS

(71) Applicants: Victor J. Johnston, Houston, TX (US); David Lee, Seabrook, TX (US); Lincoln Sarager, Houston, TX (US); R. Jay Warner, Houston, TX (US); Radmila Jevtic, Pasadena, TX (US)

(72) Inventors: Victor J. Johnston, Houston, TX (US); David Lee, Seabrook, TX (US); Lincoln Sarager, Houston, TX (US); R. Jay Warner, Houston, TX (US); Radmila Jevtic, Pasadena, TX (US)

(73) Assignee: Celanese International Corporation, Dallas, TX (US)

(21) Appl. No.: 13/273,054

(22) Filed: Oct. 13, 2011

Related U.S. Application Data
(63) Continuation of application No. PCT/US2011/046500, filed on Aug. 3, 2011.

(51) Int. Cl. C07C 29/143; C07C 31/08

(52) U.S. Cl. 568/881; 568/840

(57) ABSTRACT

A process is described for producing an alcohol product, in which (a) a first feed composition comprising acetic acid is converted to a product comprising acetone; and (b) a second feed composition comprising at least part of the acetone produced in (a) is hydrogenated in the presence of a catalyst to produce a hydrogenation effluent comprising isopropanol.

### CROSS REFERENCE TO RELATED APPLICATIONS

This application claims priority to International Patent Application No. PCT/US11/46500, filed on Aug. 3, 2011, which claims priority to U.S. Non-Provisional application Ser. No. 12/889,260, filed on Sep. 23, 2010, which claims priority to U.S. Provisional Application No. 61/300,815, filed on Feb. 2, 2010, U.S. Provisional Application No. 61/332,727, filed on May 7, 2010, and U.S. Provisional Application No. 61/332,696, filed on May 7, 2010, the entire contents and disclosures of which are incorporated herein by reference.

### FIELD OF THE INVENTION

The present invention relates to the production of alcohols and in particular to the production of isopropanol, either alone or in admixture with ethanol.

### BACKGROUND OF THE INVENTION

Isopropanol is used as a solvent and a cleaner and as a component of anti-freeze. Isopropanol can also be used as a denaturant for ethanol. Rubbing alcohol is a solution of 70% isopropanol in water. Since 1956, the annual production volume of isopropanol has exceeded one billion pounds. Isopropanol is generally produced from propylene by indirect hydration with sulfuric acid or direct hydration with water.

Ethanol is often produced for and utilized as a consumable product, e.g., beer, wine, and spirits. Typically, the ethanol for consumption is produced via conventional fermentation methods. For reasons of public policy, e.g., to inhibit the availability of inexpensive drinking alcohol, many state and national government agencies impose high duties or taxes on consumable ethanol. As a result of such taxes, the selling price of consumable ethanol is quite high, based on the cost to actually manufacture it.

There are, however, many other uses for ethanol that do not involve consumption, e.g., fuels, chemical solvents, or pharmaceuticals. As such, in an effort to provide inexpensive ethanol for non-consumable uses, most governmental agencies do not impose excessive duties or taxes on ethanol that cannot be used for consumption. Some of these non-consumable ethanols are commonly referred to as “denatured ethanols” or “denatured alcohols” and these denatured ethanols are generally prepared by adding to a denaturant pure ethanol (ethanol that has already been purified), which essentially poisons or renders undrinkable the purified ethanol. Conventional denaturants include methanol, isopropyl alcohol, acetone, methyl ethyl ketone, ethyl acetate, methyl isobutyl ketone, and actaldehyde. Thus, because these denatured ethanols are not taxed as are consumable ethanols, they are considerably less expensive to purchase.

Ethanol for industrial use is conventionally produced from petrochemical feed stocks, such as oil, natural gas, or coal, from feed stock intermediates, such as syngas, or from starchy materials or cellulose materials, such as biofuels. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic materials, are converted to ethanol by fermentation. However, fermentation is typically used for consumer production of ethanol. In addition, fermentation of starchy or cellulosic materials competes with food sources and places restraints on the amount of ethanol that can be produced for industrial use.

Ethanol production via the catalytic hydrogenation of alkanoic acids and/or other carbonyl group—containing compounds, such as acetic acid, has been widely studied, and a variety of combinations of catalysts, supports, and operating conditions have been mentioned in the literature. Acetic acid can also be converted to acetone which can undergo catalytic hydrogenation to produce iso-propanol. There is a need to produce both ethanol and isopropanol from acetic acid, both separately and as mixtures, so as, for example, to allow for the in-situ denaturing of ethanol with isopropanol.

### SUMMARY OF THE INVENTION

Accordingly, in one aspect the invention is directed to a process for producing an alcohol product, the process comprising:

(a) converting a first feed composition comprising acetic acid to a product comprising acetone; and

(b) hydrogenating a second feed composition comprising at least part of the acetone produced in (a) in the presence of a catalyst to produce a hydrogenation effluent comprising isopropanol.

Conveniently, the converting (a) is conducted by hydrogenation.

Alternatively, the converting (a) is conducted by ketonization in the presence of a metal oxide catalyst.

Conveniently, said second feed composition also comprises acetic acid.

Conveniently, the process further comprises separating an alcohol product comprising ethanol and/or isopropanol from said hydrogenation effluent.

In a further aspect, the invention is directed to ethanol denatured with in situ produced isopropanol.

### BRIEF DESCRIPTION OF THE DRAWINGS

FIG. 1 is a schematic diagram of an acetone hydrogenation system in accordance with one embodiment of the present invention.

### DETAILED DESCRIPTION OF THE EMBODIMENTS

Described herein is a process for producing an alcohol product from acetic acid, in which at least part of the acetic acid is initially converted to produce acetone. The acetone is then hydrogenated, either alone or with additional acetic acid, to produce an alcohol product comprising isopropanol, either alone or in admixture with ethanol. Isopropanol or isopropanol and ethanol can then be separately recovered from the hydrogenation effluent or a mixture of ethanol and isopropanol can be recovered, for example as an in-situ denatured ethanol product.

### Raw Materials

The raw materials, acetic acid and hydrogen, used in the present process may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the present process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used herein may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteroides, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step(s) of the present process. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Publication Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Patent No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the acetone and alcohol synthesis reaction zones of the present process without the need for condensing the acetic acid and light ends or removing water, saving overall processing costs.

### Conversion of Acetic Acid to Acetone

At least part of the acetic acid raw material used in the present process is initially fed to one or more catalytic reactors for conversion of the acetic acid to acetone.

In one embodiment. the conversion of the acetic acid to acetone is effected by partial hydrogenation according to the reaction:

2CH₃COOH+H₂→CH₃COCH₃+2H₂O+CO

The reaction can be conducted using the same catalysts and process conditions discussed below for the conversion of the acetone to isopropanol, although to inhibit complete hydrogenation of the acetone to ethanol, the molar ratio of hydrogen to acetic acid should be kept below 1:1, e.g., below 1:2. Preferred catalysts favoring acetone production include ruthenium supported by SiO₂, iron supported by carbon, and palladium supported by carbon.

In another embodiment. the conversion of the acetic acid to acetone is effected by ketonization according to the reaction:

2CH₃COOH→CH₃COCH₃+H₂O+CO₂

The reaction is generally conducted in the substantial absence of hydrogen using one or more metal oxide catalysts such as ceria, titania, zirconia, manganese oxide, tin oxide, thorium oxide and magnesium oxide. Suitable conditions for the ketonization reaction include temperatures are from about 250° C. to about 500° C. and pressures from about 5 psig to about 200 psig (135 to 1480 kPa). The main nonvolatile products of the process are water and acetone, which can be separated by simple distillation.

In either case, the conversion of the acetic acid to acetone can be conducted in any known reactor configuration, although generally a fixed bed reactor is preferred. For example, the reactor may be in the shape of a tube or pipe, where the acetic acid, generally in vapor form, is passed over or through a bed of the desired catalyst disposed in the tube or pipe. The acetic acid may be fed to the reactor in an undiluted state or diluted with a relatively inert carrier gas, such as nitrogen, argon, helium, carbon dioxide and the like.

### Conversion of Acetone to Isopropanol

The acetone produced in the preceding step is converted to isopropanol by hydrogenation according to the reaction:

CH₃COCH₃+H₂→(CH₃)₂CHOH

In some cases, the feed to the acetone hydrogenation process will include acetic acid either added as a separate feed to the acetone hydrogenation process or present as unreacted acetic acid carried over from acetone production process. Where present, the acetic acid will be converted to ethanol in the acetone hydrogenation process according to the reaction:

CH₃COOH+2H₂→CH₃CH₂OH+H₂O

The hydrogenation of the acetone either alone or in the presence of acetic acid is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Exemplary catalysts are further described in U.S. Pat. No. 7,608,744 and U.S. Publication No. 2010/0029995, the entireties of which are incorporated herein by reference. In another embodiment, the catalyst comprises a Co/Mo/S catalyst of the type described in U.S. Publication No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of the desired alcohol product.

Suitable support materials may include, for example, stable metal oxide-based supports or ceramic-based supports. Preferred supports include siliceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group JIB metal oxides, (vi) Group JIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain NorPro. The Saint-Gobain NorPro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present process preferably are formed through metal impregnation of the modified support, although other processes such as chemical vapor deposition may also be employed. Such impregnation techniques are described in U.S. Pat. Nos. 7,608,744 and 7,863,489 and U.S. Publication No. 2010/0197485 referred to above, the entireties of which are incorporated herein by reference.

In particular, the hydrogenation of acetone by the present process may achieve favorable conversion of acetone and favorable selectivity and productivity to isopropanol. For purposes of the present invention, the term “conversion” refers to the amount of acetone in the feed that is converted to a compound other than acetone. Conversion is expressed as a mole percentage based on acetone in the feed. The conversion may be at least 10%, e.g., at least 20%, at least 40%, at least 50%, at least 60%, at least 70% or at least 80%. Similar, percentages are obtained in the conversion of mixed acetone/acetic acid feeds. Although catalysts that have high conversions are desirable, such as at least 80% or at least 90%, in some embodiments a low conversion may be acceptable at high selectivity for isopropanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetone or converted acetone and acetic acid. It should be understood that each compound converted from acetone or acetic acid has an independent selectivity and that selectivity is independent from conversion. For example, if 60 mole % of the converted acetone is converted to isopropanol, we refer to the isopropanol selectivity as 60%. Preferably, the selectivity to isopropanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetone passed over the catalyst is converted to alkanes, which have little value other than as fuel.

The term “productivity,” as used herein, refers to the grams of a specified product, e.g., isopropanol, formed during the hydrogenation based on the kilograms of catalyst used per hour. A productivity of at least 100 grams of isopropanol per kilogram of catalyst per hour, e.g., at least 400 grams of isopropanol per kilogram of catalyst per hour or at least 600 grams of isopropanol per kilogram of catalyst per hour, is preferred. In terms of ranges, the productivity preferably is from 100 to 3,000 grams of isopropanol per kilogram of catalyst per hour, e.g., from 400 to 2,500 grams of isopropanol per kilogram of catalyst per hour or from 600 to 2,000 grams of isopropanol per kilogram of catalyst per hour.

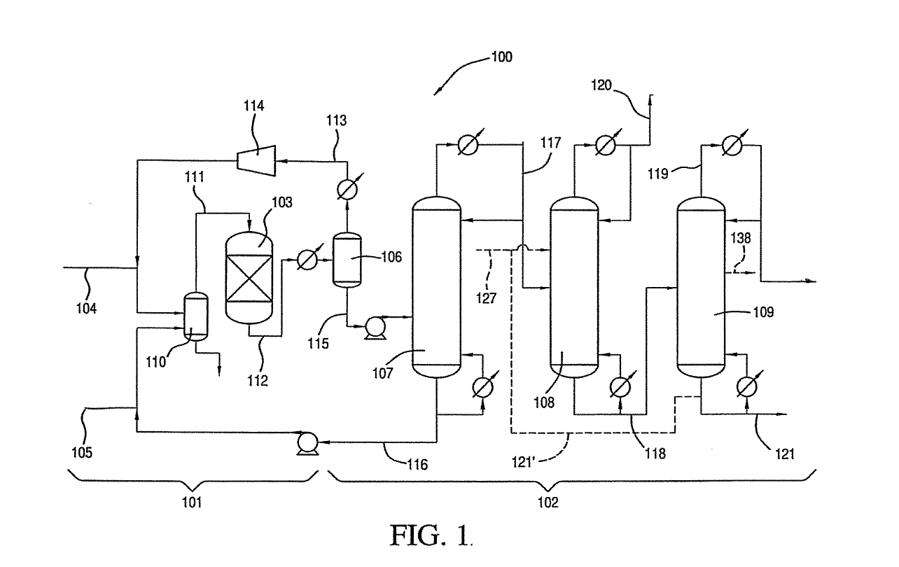

Referring to the drawings, FIG. 1 shows a hydrogenation system 100 suitable for the hydrogenation of acetone and separating isopropanol from the crude reaction product according to one embodiment of the invention. The upstream part of the process, where acetic acid is converted into acetone is not shown in FIG. 1.

System 100 comprises a reaction zone 101 and a purification zone 102. Reaction zone 101 comprises reactor 103, hydrogen feed line 104 and acetone feed line 105. In other embodiments, where acetic acid is also utilized as a reactant, reaction zone 101 further comprises an acetic acid feed line (not shown). Purification zone 102 comprises separator 106, first distillation column 107, second distillation column 108, and third distillation column 109. Hydrogen, acetone, and optionally acetic acid are fed to a vaporizer 110 via lines 104 and 105, respectively, to create a vapor feed stream in line 111 that is directed to reactor 103. In one embodiment, lines 104 and 105 may be combined and jointly fed to the vaporizer 110, e.g., in one stream containing both hydrogen and acetone. The temperature of the vapor feed stream in line 111 is preferably from 100° C. to 350° C., e.g., from 120° C. to 310° C. or from 150° C. to 300° C. Any feed that is not vaporized is removed from vaporizer 110, as shown in FIG. 1, and may be recycled thereto. In addition, although FIG. 1 shows line 111 being directed to the top of reactor 103, line 111 may be directed to the side, upper portion, or bottom of reactor 103. Although one reactor and one flasher are shown in FIG. 1, additional reactors and/or components may be included in various optional embodiments of the present invention. For example, the hydrogenation system may optionally comprise dual reactors, dual flashers, heat exchanger(s), and/or pre-heater(s).

In one embodiment, one or more guard beds (not shown) may be used upstream of the reactor, optionally upstream of the vaporizer 110, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a crude hydrogenation product stream is withdrawn, preferably continuously, from reactor 103 via line 112.

The crude hydrogenation product stream in line 112 may be condensed and fed to the separator 106, which, in turn, provides a vapor stream 113 and a liquid stream 115. In some embodiments, separator 106 may comprise a flasher or a knockout pot. Separator 106 may operated at a temperature of from 20° C. to 250° C., e.g., from 30° C. to 225° C. or from 60° C. to 200° C. The pressure of separator 106 may be from 50 kPa to 2000 kPa, e.g., from 75 kPa to 1500 kPa or from 100 kPa to 1000 kPa. Optionally, the crude hydrogenation product in line 112 may pass through one or more membranes to separate hydrogen and/or other non-condensable gases.

The vapor stream exiting the separator 106 may comprise hydrogen and hydrocarbons, which may be purged and/or returned to reaction zone 103 via line 113. As shown in FIG. 1, the returned portion of the vapor stream passes through compressor 114 and is combined with the hydrogen feed and co-fed to vaporizer 110.

The liquid stream 115 is withdrawn from separator 106 and pumped to the side of first distillation column 107, which is also referred to as the acid separation column. The contents of line 115 typically will be substantially similar to the product obtained directly from the reactor, and may, in fact, also be characterized as a crude hydrogenation product. However, the liquid stream 115 preferably has substantially no hydrogen, carbon dioxide, methane or ethane, which are removed by separator 106. The principal components of the liquid stream are isopropanol, unreacted acetone and, where acetic acid is included in the feed to the reactor 103, ethanol, water and unreacted acetic acid. In fact, if the content of acetic acid in line 115 is less than 5 wt. %, the acid separation column 107 may be skipped and line 115 may be introduced directly to second column 108, also referred to herein as a light ends column.

In the first distillation column 107, unreacted acetic acid, a portion of the water, and other heavy components, if present, are removed from the composition in line 115 and are withdrawn, preferably continuously, as residue. Some or all of the residue may be returned and/or recycled back to reaction zone 103 via line 116. First column 107 also forms an overhead distillate, which is withdrawn in line 117, and which may be condensed and refluxed, for example, at a ratio of from 10:1 to 1:10, e.g., from 3:1 to 1:3 or from 1:2 to 2:1.

Any of columns 107, 108 or 109 may comprise any distillation column capable of separation and/or purification. The columns preferably comprise tray columns having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The associated condensers and liquid separation vessels that may be employed with each of the distillation columns may be of any conventional design and are simplified in FIG. 1. As shown in FIG. 1, heat may be supplied to the base of each column or to a circulating bottom stream through a heat exchanger or reboiler. Other types of reboilers, such as internal reboilers, may also be used in some embodiments. The heat that is provided to reboilers may be derived from any heat generated during the process that is integrated with the reboilers or from an external source such as another heat generating chemical process or a boiler. Although one reactor and one flasher are shown in FIG. 1, additional reactors, flashers, condensers, heating elements, and other components may be used in embodiments of the present invention. As will be recognized by those skilled in the art, various condensers, pumps, compressors, reboilers, drums, valves, connectors, separation vessels, etc., normally employed in carrying out chemical processes may also be combined and employed in the present process.

The temperatures and pressures employed in any of the columns 107 to 109 may vary. As a practical matter, pressures from 10 kPa to 3000 kPa will generally be employed in these zones although in some embodiments subatmospheric pressures may be employed as well as superatmospheric pressures. Temperatures within the various zones will normally range between the boiling points of the composition removed as the distillate and the composition removed as the residue. It will be recognized by those skilled in the art that the temperature at a given location in an operating distillation column is dependent on the composition of the material at that location and the pressure of column. In addition, feed rates may vary depending on the size of the production process and, if described, may be generically referred to in terms of feed weight ratios.

When column 107 is operated under standard atmospheric pressure, the temperature of the residue exiting in line 116 from column 107 preferably is from 95° C. to 120° C., e.g., from 105° C. to 117° C. or from 110° C. to 115° C. The temperature of the distillate exiting in line 117 from column 107 preferably is from 70° C. to 110° C., e.g., from 75° C. to 95° C. or from 80° C. to 90° C. In other embodiments, the pressure of first column 107 may range from 0.1 kPa to 510 kPa, e.g., from 1 kPa to 475 kPa or from 1 kPa to 375 kPa.

The first column distillate in line 117 comprises isopropanol, unreacted acetone and, where acetic acid is included in the feed to the reactor 103, ethanol, water and other impurities, such as ethyl acetate, which may be difficult to separate due to the formation of binary and tertiary azeotropes. The first distillate comprises a significantly reduced amount of acetic acid.

The first distillate in line 117 is introduced to the second column 108, also referred to as the “light ends column,” preferably in the middle part of column 108, e.g., middle half or middle third. Second column 108 may be a tray column or packed column. In one embodiment, second column 108 is a tray column having from 5 to 70 trays, e.g., from 15 to 50 trays or from 20 to 45 trays.

As one example, when a 25 tray column is utilized in a column without water extraction, line 117 is introduced at tray 17. When the second column is not an extractive distillation column, it is expected that any ethyl acetate in line 117 may be separated into the residue of the second column 108 along with the isopropanol, ethanol and water. More preferably, the second column 108 is an extractive distillation column, in which case it is expected that any ethyl acetate in line 117 may be separated from the isopropanol, ethanol and water and pass into the distillate of the second column 108. In such embodiments, an extraction agent, such as water, may be optionally added to second column 108 via line 127. If the extraction agent comprises water, it may be obtained from an external source or from an internal return/recycle line from one or more of the other columns. In a preferred embodiment, the water in the third residue of third column 109 is utilized as the extraction agent. As shown in FIG. 1, the third residue may be optionally directed to second column 108 via line 121′.

Although the temperature and pressure of second column 108 may vary, when at atmospheric pressure, the temperature of the second column residue is typically from 60° C. to 90° C., e.g., from 70° C. to 90° C. or from 80° C. to 90° C., while the temperature of the second column distillate is typically from 50° C. to 90° C., e.g., from 60° C. to 80° C. or from 60° C. to 70° C. Column 108 may operate at atmospheric pressure. In other embodiments, the pressure of second column 108 may range from 0.1 kPa to 510 kPa, e.g., from 1 kPa to 475 kPa or from 1 kPa to 375 kPa.

The second column distillate exits the column 108 via line 120 and mostly comprises unreacted acetone, which can be recycled to the reactor 103. Any ethyl acetate in line 120 can be separated from the acetone and supplied to the purified alcohol product exiting column 109 in distillate line 119, for example to act as an additional denaturant to an ethanol or ethanol/isopropanol mixture in the distillate line 119.

The second column residue comprises isopropanol, optionally with ethanol and water, and is fed via line 118 to third column 109, also referred to as the “product column.” More preferably, the second residue in line 118 is introduced in the lower part of third column 109, e.g., the lower half or lower third. Third column 109 is preferably a tray column as described above and preferably operates at atmospheric pressure. The temperature of the distillate exiting the top of the third column 109 in line 119 is typically from 60° C. to 110° C., e.g., from 70° C. to 100° C. or from 75° C. to 95° C. The temperature of the residue exiting from third column 109 in line 121 is typically from 70° C. to 115° C., e.g., from 80° C. to 110° C. or from 85° C. to 105° C., when the column is operated at atmospheric pressure. The distillate in line 119 is preferably refluxed as shown in FIG. 1, for example, at a reflux ratio of from 1:10 to 10:1, e.g., from 1:3 to 3:1 or from 1:2 to 2:1

The third column residue in line 121 comprises primarily water, which may be removed from the system 100 or may be partially returned to any portion of the system 100, e.g., via line 121′.

The third column 109 may be constructed and operated to recover a single alcohol product as the distillate in line 119 exiting the top of the column. In such a case, the single alcohol product is composed mainly of isopropanol or a mixture of isopropanol and ethanol, which is preferably substantially pure other than the azeotropic water content. The isopropanol product can be sold directly or after further purification, while the mixture of isopropanol and ethanol can be sold as denatured ethanol, either directly or after mixing with additional ethanol, or can be used as a fuel additive.

Alternatively, the third column 109 may be operated to recover a first distillate product in the line 119 exiting the top of the column and one or more higher-boiling distillate products in side streams 138 exiting the column 109 from a middle or an upper section of third column. Since isopropanol has a higher boiling point (82.5° C.) than that of ethanol (78° C.), an ethanol rich stream can be recovered by way of line 119 and an isopropanol-rich stream can be recovered by way of side stream 138. Both alcohol streams can be sold directly or after further purification

1. A process for producing an alcohol product, the process comprising:
(a) converting a first feed composition comprising acetic acid in a hydrogenation reactor in the presence of a first catalyst selected from the group consisting of ruthenium supported by SiO₂, iron supported by carbon, and palladium supported by carbon to produce a product comprising acetone;
(b) hydrogenating a second feed composition comprising at least part of the acetone produced in (a) in the presence of a second catalyst in a second reactor to produce a hydrogenation effluent comprising isopropanol and acetone, wherein the second catalyst comprises at least a first metal and a second metal, wherein the second metal is different from the first metal; and
(c) separating at least a portion of the acetone from the isopropanol and returning the acetone to the second reactor.

2. (canceled)

3. The process of claim 1, wherein the hydrogenation (a) is conducted at a molar ratio of hydrogen to acetic acid of below 1:1, and preferably below 1:2.

4. The process of claim 1, wherein the first metal is on a support.

5. The process of claim 1, wherein the first metal is selected from the group consisting of Group IB, IIB, IIIB, IVB, VB, VIB, VIIB, VIII transition metals, a lanthanide metal, an actinide metal or a metal selected from any of Groups IIIA, IVA, VA, and VIA.

6. The process of claim 4, wherein the support is selected from the group consisting of silica, silica/alumina, calcium metasilicate, iron oxide, alumina, titania, zirconia, magnesium oxide, carbon, graphite, graphitized carbon, activated carbons, and mixtures thereof.

7. (canceled)

8. The process of claim 1, wherein the second metal is selected from the group consisting of copper, molybdenum, tin, chromium, iron, cobalt, vanadium, tungsten, palladium, platinum, lanthanum, cerium, manganese, ruthenium, rhenium, gold, and nickel.

9. (canceled)

10. (canceled)

11. (canceled)

12. (canceled)

13. The process of claim 22, wherein the first metal is selected from the group consisting of Group IB, IIB, IIIB, IVB, VB, VIB, VIIB, VIII transition metals, a lanthanide metal, an actinide metal or a metal selected from any of Groups IIIA, IVA, VA, and VIA.

14. The process of claim 22, wherein the support is selected from the group consisting of silica, silica/alumina, calcium metasilicate, iron oxide, alumina, titania, zirconia, magnesium oxide, carbon, graphite, graphitized carbon, activated carbons, and mixtures thereof.

15. (canceled)

16. The process of claim 22, wherein the second metal is selected from the group consisting of copper, molybdenum, tin, chromium, iron, cobalt, vanadium, tungsten, palladium, platinum, lanthanum, cerium, manganese, ruthenium, rhenium, gold, and nickel.

17. The process of claim 1, further comprising separating at least one alcohol product comprising isopropanol from said hydrogenation effluent.

18. The process of claim 1, wherein the second feed composition also comprises acetic acid.

19. The process of claim 18, further comprising separating at least one alcohol product comprising ethanol and/or isopropanol from said hydrogenation effluent.

20. (canceled)

21. The process of claim 1, wherein the second catalyst is selected from the group consisting of platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron.

22. A process for producing an alcohol product, the process comprising:
(a) converting a first feed composition comprising acetic acid in a ketonization reactor in the presence of a metal oxide catalyst selected from the group consisting of ceria, titania, zirconia, manganese oxide, tin oxide, thorium oxide, magnesium oxide and mixtures thereof to produce a product comprising acetone;
(b) hydrogenating a second feed composition comprising at least part of the acetone produced in (a) in the presence of a second catalyst in a second reactor to produce a hydrogenation effluent comprising isopropanol and acetone, wherein the second catalyst comprises at least a first metal on a support and a second metal, wherein the second metal is different from the first metal; and
(c) separating at least a portion of the acetone from the isopropanol and returning the acetone to the second reactor.

23. The process of claim 22, further comprising separating at least one alcohol product comprising isopropanol from the hydrogenation effluent.

24. The process of claim 23, further comprising separating at least one alcohol product comprising ethanol and/or isopropanol from the hydrogenation effluent.

25. The process of claim 22, wherein the second feed composition also comprises acetic acid.

26. The process of claim 22, wherein the second catalyst is selected from the group consisting of platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron.
